(19) United States
(12) Patent Application Publication
(10) Pub. No.: US 2018/0335434 A1
(43) Pub. Date: Nov. 22, 2018

(54) Methods of Detecting Circulating Tumor Cells

(71) Applicants: HITACHI CHEMICAL COMPANY, LTD., Tokyo (JP); National University Corporation UNIVERSITY OF FUKUI, Fukui-shi (JP)

(72) Inventors: Katsuya Endou, Tokyo (JP); Hisashige Kanbara, Tokyo (JP); Tatsuya Matsunaga, Tokyo (JP); Yasunari Nakamoto, Yoshida-gun (JP); Kazuto Takahashi, Yoshida-gun (JP); Kazuya Ofuji, Yoshida-gun (JP); Katsushi Hiramatsu, Yoshida-gun (JP); Takuto Nosaka, Yoshida-gun (JP)

(73) Assignees: HITACHI CHEMICAL COMPANY, LTD., Tokyo (JP); National University Corporation UNIVERSITY OF FUKUI, Fukui-shi (JP)

(21) Appl. No.: 15/981,397

(22) Filed: May 16, 2018

Related U.S. Application Data
(60) Provisional application No. 62/507,770, filed on May 17, 2017.

(51) Int. Cl. G01N 33/574

(52) U.S. Cl. CPC G01N 33/57438

(57) ABSTRACT

The disclosure relates to a method of detecting circulating tumor cells (CTCs) in a subject, diagnosing cancer, and/or treating a subject in need thereof.

### BACKGROUND

The disclosure relates to a method of detecting circulating tumor cells (CTCs) in a subject, diagnosing cancer, and/or treating a subject in need thereof.

### SUMMARY

The disclosure relates to a method of treating a subject without a detectable solid tumor comprising: detecting circulating tumor cells (CTCs) from a blood sample from the subject; and treating the subject with chemotherapy or immunotherapy for cancer.

The disclosure also relates to a method of decreasing a number of CTC in a subject without a detectable solid tumor comprising: detecting circulating tumor cells (CTCs) from a blood sample from the subject; and decreasing the number of CTC in the subject by chemotherapy or immunotherapy for cancer.

The disclosure further relates to a method of treating liver cancer in a subject in need thereof comprising: detecting circulating tumor cells (CTCs) from a blood sample from a subject without a detectable solid tumor; and treating the liver cancer in the subject without a detectable solid tumor.

The disclosure also relates to a method of diagnosing cancer in a subject, comprising detecting circulating tumor cells (CTCs) from a blood sample from a subject, and determining whether the subject has cancer based on the concentration or number of CTCs in the blood sample. The disclosure further relates to a method of treating cancer in the subject that is determined to have cancer. Additionally, the disclosure relates to a device or system used to diagnose cancer in accordance with the methods described herein.

### BRIEF DESCRIPTION OF DRAWINGS

FIG. 1 depicts a scheme of an exemplary microcavity array system.

FIG. 2 depicts exemplary CTC recovery rates.

FIGS. 3 and 4 depict patient data and CTC count results.

### DETAILED DESCRIPTION

As hematogenous spread is the major route of HCC metastasis and recurrence, detection of circulating tumor cells (CTCs) has important clinical significance in HCC patients. Recent reports indicated that CTCs were entrapped from unprocessed human whole blood based on differences in the size and deformability between tumor cells and other blood cells using a microcavity array (MCA) system.

In one aspect, the disclosure relates to a method of treating a subject without a detectable solid tumor comprising: detecting circulating tumor cells (CTCs) from a blood sample from the subject; and treating the subject with chemotherapy or immunotherapy for cancer. In another aspect, the disclosure also relates to a method of decreasing a number of CTC in a subject without a detectable solid tumor comprising: detecting circulating tumor cells (CTCs) from a blood sample from the subject; and decreasing the number of CTC in the subject by chemotherapy or immunotherapy for cancer. In another aspect, the disclosure further relates to a method of treating liver cancer in a subject in need thereof comprising: detecting circulating tumor cells (CTCs) from a blood sample from a subject without a detectable solid tumor; and treating the liver cancer in the subject without a detectable solid tumor. In another aspect, the disclosure also relates to a method of diagnosing cancer in a subject, comprising detecting circulating tumor cells (CTCs) from a blood sample from a subject, and determining whether the subject has cancer based on the concentration or number of CTCs in the blood sample. The disclosure further relates to a method of treating cancer in the subject that is determined to have cancer.

The “solid tumor” herein means a solid mass of cancer cells from which a piece of tissue can be removed for biopsy. The tumor or cancer described herein may include bile duct cancer, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), gall bladder cancer, bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, lung cancer, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypemephroma, insulinoma, islet tumor, keratoma, leiomyoblastoma, leiomyosarcoma, lymph node metastatic cancer, lymphosarcoma, melanoma, mammary tumor, mastocytoma, medulloblastoma, malignant mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma. The “detectable solid tumor” herein means a solid tumor that can be detectable with a known method in the field of cancer diagnostics at the time of filing this application, without detecting CTCs. For example, such known methods to detect solid tumor includes biopsy and imaging, such as computed tomography (CT) scans, magnetic resonance imaging (Mill) scans, ultrasound, positron emission tomography (PET) scan, and X-ray imaging. For example, the tumor or cancer described herein may be liver cancer, and the detectable solid tumor cell may be detectable liver tumor. The “circulating tumor cell” (CTC) is a cancer cell that detach from a primary tumor and travel though blood stream or lymphatic system to other parts of the body. The CTCs may be detected by methods described in U.S. Patent Application Publication Nos. 2014-0178890; 2014-0238863; 2015-0004687; 2014-0299539; 2015-0111293; 2016-0195458; and 2016-0169781, all of which are herein incorporated by reference in their entirety. The “chemotherapy” described herein is a use of a drug to treat a cancer or tumor. The chemotherapy may include any known drug therapy to treat cancer, for example, including the therapy described in U.S. Patent Application Publication No. 2010/0015042, all of which contents are herein incorporated by reference in its entirety. The chemotherapy may include neoadjuvant chemotherapy that refers to a use of a drug before deciding to proceed with a surgery for cancer treatment. The surgery may be the first surgery for the subject, or another surgery after recurrence of cancer in the subject. The “immunotherapy for cancer” described herein is a use of a substance that stimulate the immune response to treat a cancer or tumor. The immunotherapy herein may include any known immunotherapy to treat cancer, for example, including a use of any the immunomodulating agents described in U.S. Patent Application Publication No. 2007/0081972, which is herein incorporated by reference in its entirety.

In some embodiments, the “subject” described herein is a patient having a chronic liver disease. In additional embodiments, the tumor or cancer in such a subject may be liver cancer. In further embodiments, the subject described herein has not been diagnosed with cancer.

In some embodiments, the method may comprise collecting the blood sample from the subject or the patient described herein. In further embodiments, the blood sample has at least 1, 2, 3, 4, 5, 6, 7, 8, 9, 10, 11, 12, 13, 14, 15, 16, 17, 18, 19 and 20 mL of whole blood from the subject or the patient. In additional embodiments, the method may comprise diagnosing whether the subject has cancer.

In some embodiments, the detecting described herein comprises determining a concentration of CTCs from the blood sample. In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 60, 65, 70 or 80 CTCs are detected per 3 mL of the blood sample or of the whole blood of the subject. In additional embodiments, 90, 80, 70, 60, 50, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4 or 3 or fewer CTCs are detected per 3 mL of the blood sample or of the whole blood of the subject. In further embodiments, 1-20, 2-20, 3-15, 4-15, 5-15, 6-15, 7-15 or 8-15 CTCs are detected per 3 mL of the blood sample or of the whole blood of the subject.

In some embodiments, the detecting comprises trapping the CTCs using a filter. The filter herein includes the filters described in U.S. Patent Application Publication Nos. 2014-0178890; 2014-0238863; 2015-0004687; 2014-0299539; 2015-0111293; 2016-0195458; and 2016-0169781, all of which are herein incorporated by reference in their entirety. In some embodiments, the detecting comprises trapping at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50 or 60 CTCs in the filter. In additional embodiments, the detecting comprises trapping 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4 or 3 or fewer CTCs in the filter. In further embodiments, the detecting comprises trapping 1-100, 2-50, 3-30, 4-20, 5-20, 6-20, 7-20 or 8-20 CTCs in the filter.

In another aspect, as described herein, the method of treating liver cancer in a subject in need thereof may comprising detecting circulating tumor cells (CTCs) from a blood sample from a subject without a detectable solid tumor; and treating the liver cancer in the subject without a detectable solid tumor. In some embodiments, the liver cancer in a patient may be treated by chemotherapy, immunotherapy, radiation therapy, hormone therapy, or any other cancer therapy known in the art.

Additionally, the disclosure relates to a device or system used to diagnose cancer in accordance with the methods described herein. The device or system used to detecting the CTCs may be the device or system described in U.S. Patent Application Publication Nos. 2014-0178890; 2014-0238863; 2015-0004687; 2014-0299539; 2015-0111293; 2016-0195458; and 2016-0169781, all of which are herein incorporated by reference in their entirety.

The following examples are illustrative and are not intended to limit the scope of the invention described herein.

### Example 1

From January 2015 to January 2016, peripheral blood samples were collected from 19 patients with HCC, 11 patients with chronic liver diseases (CLDs) without any cancers, and 7 healthy volunteers.

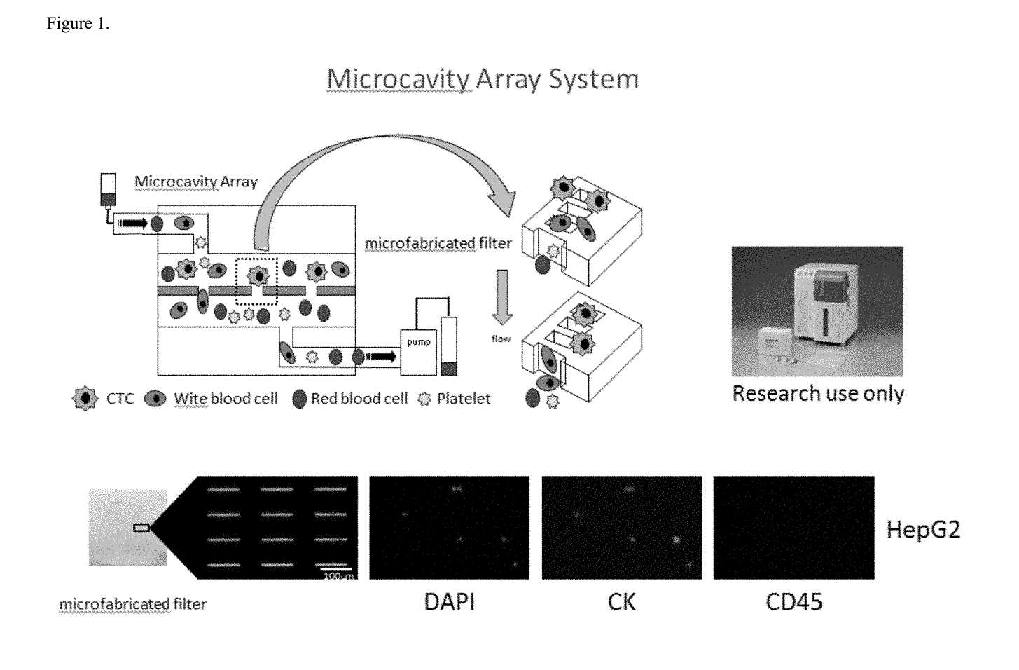

To enrich CTCs from whole blood, a microfabricated filter with a rectangular MCA was integrated with a miniaturized device. The shape and porosity of the MCA were optimized to efficiently capture tumor cells on the microcavities under low flow resistance condition, while allowing other blood cells to effectively pass through. The scheme of the microcavity array system is shown in FIG. 1.

Isolated CTCs that were stained immunochemically with cytokeratin (CK), DAPI and CD45 were analyzed by fluorescent microscopy. We defined cells characterized by immunofluorescent intensities that were positive for cytokeratin and DAPI and negative for CD45 as CTC.

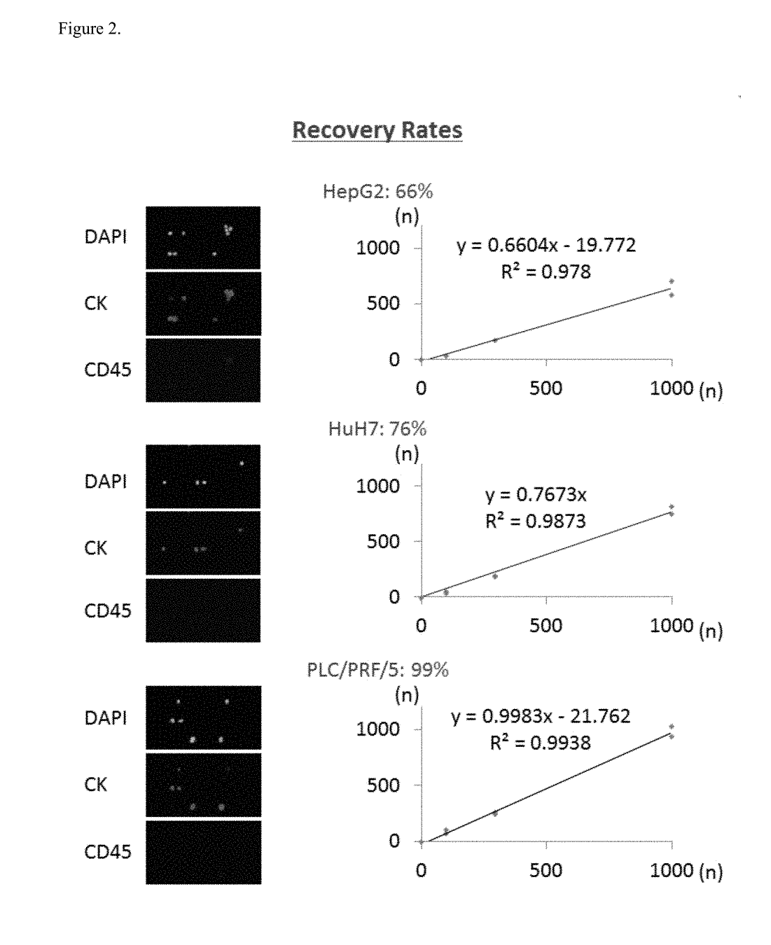

Firstly, we investigated recovery of spiked HepG2, HuH7 and PLC/PRF/5 cells that were human hepatoma cell lines from whole blood using the MCA system. Those cells (0, 100, 300, and 1000 cells) were spiked into 3 ml of blood from a healthy volunteer. The results of the recovery rate are shown in FIG. 2.

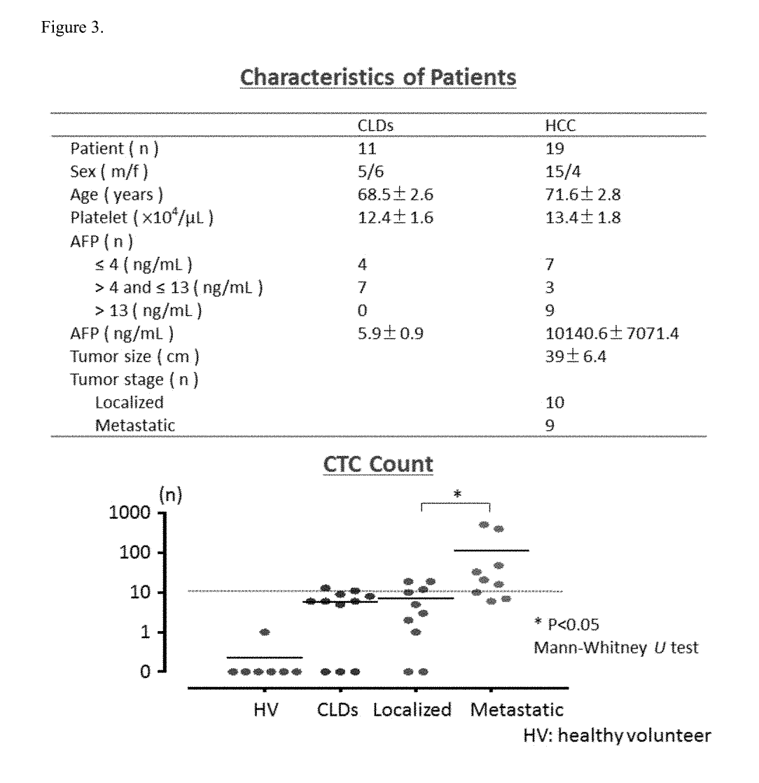

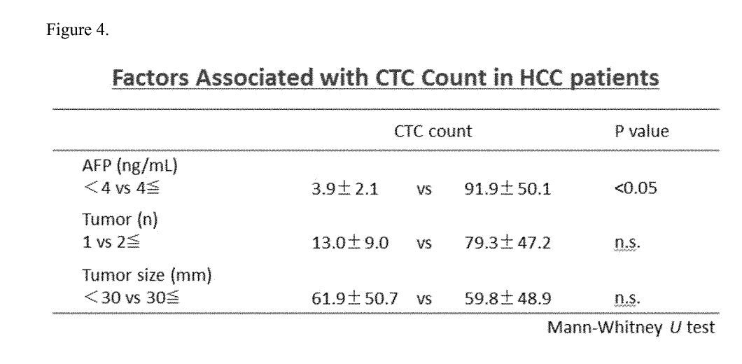

In the samples (3 mL whole blood) of patients with HCC, the mean number of detected CTCs was 59.5±32.7 (mean±SE), and the positivity rate of patients (CTCs>10 cells) was 47.3% (9/19). In contrast, the mean number of CTCs was 5.8±1.3 and the positivity rate was 18.1% (2/11) in the patients with CLDs without HCC. Furthermore, the numbers of CTCs were significantly increased in the patients with vascular invasion and metastasis of HCC, compared to those with localized HCC (117.7±65.4 v.s. 7.1±2.4; p<0.05). The results are shown in FIGS. 3 and 4.

Conclusion: The MCA system has a potential to isolate CTCs at high recovery rates in HCC cell lines and at high sensitivity in the patients. The numbers of CTCs are correlated with the progression of HCC, in which more numbers of CTCs are seen in the patients with vascular invasion and metastasis of tumor. The results suggest that the MCA system may provide a new strategy for high detection rates of CTCs and a predictive tool for the clinical risk of extrahepatic progression of HCC.

### Example 2

From January 2015 to January 2016, peripheral blood samples were collected from 19 patients with HCC, 11 patients with chronic liver diseases (CLDs) without any cancers, and 7 healthy volunteers. To enrich CTCs from whole blood, a microfabricated filter with a rectangular MCA was integrated with a miniaturized device. The shape and porosity of the MCA were optimized to efficiently capture tumor cells on the microcavities under low flow resistance condition, while allowing other blood cells to effectively pass through. Isolated CTCs that was stained immunochemically with cytokeratin, DAPI and CD45 was analyzed by fluorescent microscopy. We defined cells characterized by immunofluorescent intensities that were positive for cytokeratin and DAPI and negative for CD45 as CTC.

The average recovery rates of HepG2, HuH7 and PLC/PRF/5 cells using the MCA system were 66%, 76% and 99%, respectively. In the samples (3 mL whole blood) of patients with HCC, the mean number of detected CTCs was 59.5±32.7 (mean±SE), and the positivity rate of patients (CTCs>10 cells) was 47.3% (9/19). In contrast, the mean number of CTCs was 5.8±1.3 and the positivity rate was 18.1% (2/11) in the patients with CLDs without HCC. None of 7 healthy volunteers showed positive. Furthermore, the numbers of CTCs were significantly increased in the patients with vascular invasion and metastasis of HCC, compared to those with localized HCC (117.7±65.4 v.s. 7.1±2.4; p<0.05). The numbers of CTCs detected in patients with serum AFP≥4 ng/ml were significantly higher than those in patients with serum AFP<4 ng/ml (91.9±50.1 v.s. 3.9±2.1; p<0.05).

The MCA system has a potential to isolate CTCs at high recovery rates in HCC cell lines and at high sensitivity in the patients. The numbers of CTCs are correlated with the progression of HCC, in which more numbers of CTCs are seen in the patients with vascular invasion and metastasis of tumor. The results suggest that the MCA system may provide a new strategy for high detection rates of CTCs and a predictive tool for the clinical risk of extrahepatic progression of HCC.

1. A method of treating a subject without a detectable solid tumor comprising,
detecting at least 2 circulating tumor cells (CTCs) from a blood sample from the subject; and
treating the subject with chemotherapy or immunotherapy for cancer.

2. The method according to claim 1, wherein the subject is a patient having a chronic liver disease.

3-7. (canceled)

8. The method according to claim 1, wherein at least 3 CTCs were detected per 3 mL whole blood of the subject.

9. The method according to claim 1, wherein at least 10 CTCs were detected per 3 mL whole blood of the subject.

10-11. (canceled)

12. The method according to claim 1, wherein the detecting comprises trapping the CTCs using a filter.

13-15. (canceled)

16. A method of decreasing a number of CTC in a subject without a detectable solid tumor comprising,
detecting circulating tumor cells (CTCs) from a blood sample from the subject; and
decreasing the number of CTC in the subject by chemotherapy or immunotherapy for cancer.

17. The method according to claim 16, wherein the subject is a patient having a chronic liver disease.

18-22. (canceled)

23. The method according to claim 16, wherein at least 3 CTCs were detected per 3 mL whole blood of the subject.

24. The method according to claim 16, wherein at least 10 CTCs were detected per 3 mL whole blood of the subject.

25-26. (canceled)

27. The method according to claim 16, wherein the detecting comprises trapping the CTCs using a filter.

28-30. (canceled)

31. A method of treating liver cancer in a subject in need thereof comprising,
detecting circulating tumor cells (CTCs) from a blood sample from a subject without a detectable solid tumor; and
treating the liver cancer in the subject without a detectable solid tumor.

32. The method according to claim 31, wherein the subject is a patient having a chronic liver disease.

33-38. (canceled)

39. The method according to claim 31, wherein at least 3 CTCs were detected per 3 mL whole blood of the subject.

40. The method according to claim 31, wherein at least 10 CTCs were detected per 3 mL whole blood of the subject.

41-42. (canceled)

43. The method according to claim 31, wherein the detecting comprises trapping the CTCs using a filter.

44-46. (canceled)
